(19) United States
(12) Patent Application Publication
(10) Pub. No.: US 2014/0065067 A1
(43) Pub. Date: Mar. 6, 2014

(54) RADIOPAQUE IODINATED PRODUCTS INTENDED FOR USE MEDICAL IMAGING AND THEIR METHODS

(71) Applicants: François Hallouard, Lyon (FR); Nicolas Anton, Illkirch-Graffenstaden (FR); Guy Zuber, Illkirch-Graffenstaden (FR); Thierry Vandamme, Strasbourg (FR); André Constantinesco, Strasbourg (FR); Philippe Choquet, Rorschwihr (FR)

(72) Inventors: François Hallouard, Lyon (FR); Nicolas Anton, Illkirch-Graffenstaden (FR); Guy Zuber, Illkirch-Graffenstaden (FR); Thierry Vandamme, Strasbourg (FR); André Constantinesco, Strasbourg (FR); Philippe Choquet, Rorschwihr (FR)

(73) Assignee: UNIVERSITE DE STRASBOURG, Strasbourg Cedex (FR)

(21) Appl. No.: 13/994,250

(22) PCT Filed: Dec. 13, 2011
(86) PCT No.: PCT/EP11/72655

§ 371 Date: Nov. 20, 2013

(51) Int. Cl. A61K 51/04; A61K 49/00

(52) U.S. Cl. CPC A61K 51/0402; A61K 49/0004
USPC 424/1.65; 560/230; 424/9.1

(57) ABSTRACT

Iodinated amphiphilic compound of formula (I):

AG₁-X—R (I)

wherein AG₁ represents an unsaturated (C₈-C₅₂) aliphatic hydrocarbon chain found in fatty acids having specific location of double bonds, the chain including at least one iodine atom directly linked to a carbon atom of a non-conjugated double bond by a covalent link, the second carbon atom of the double bond bearing a halogen atom different from a iodine atom, X represents a polyethylene glycol including 1 to 50 ethylene glycol groups, R represents a hydrogen atom or a group AG₂ being identical to or different from AG₁, AG₁, X, R and AG₂ being selected so that the amphiphilic compound has a hydrophilic/lipohilic balance included between 4 and 30.

### SUMMARY OF THE INVENTION

The invention refers to the formulation of injectable nano-emulsions containing iodinated amphiphilic agents intended for medical imaging in animals and humans. The nano-emulsions are prepared by a low energy process. The iodinated amphiphilic agents used contain iodine atoms directly fixed on the aliphatic backbone and are prepared using simple chemical processes.

### DESCRIPTION OF THE INVENTION

Among the various progresses realized in the medical field during the last decades, it is advisable to resort to medical imaging to visualize the interior of a living organism by noninvasive way. These recent discoveries allow not only a better diagnosis but also offer new hopes for the treatment of many diseases such as cancer, heart-dysfunctions, etc. . . . Indeed medical imaging open doors to detect and identify tumors and lesions precisely and thus facilitate surgical operations, the only therapeutic solution for some patients. Moreover, these techniques allow better understanding of the function and operation of highly complicated and still mysterious (largely unexplored) organs like the brain.

The medical imaging gathers all the means of acquisition and restitution of images starting from various physical phenomena (Magnetic resonance, reflection of waves ultrasounds, radioactivity, absorption of the X-rays, . . . ). The use of these imaging techniques indirectly allows visualizing the physiology or the metabolism of the human body or other animals. The image obtained will make it possible (for example) to show the evolution or the movements of a substance in the living tissue in the course of time, to give a three-dimensional reconstitution of an organ or tissue. The quantitative images also represent the values measured for certain biological parameters in a given volume.

Depending on the technique used, the interpretation of medical imaging data, makes it possible to obtain information on the anatomy of the organs (size, volume, localization, the form of a possible lesion, etc) or on their operation (physiology, metabolism, etc). In the first case one speaks about structural imaging and in the second one of functional imaging.

Among the commonly used methods of structural imaging in medicine, one can note the tomographic methods based on the X-rays (conventional radiology, tomodensitometer or CT-scan, angiography, . . . ) maybe on magnetic resonance (MRI), echographic methods (which use the ultrasounds), and finally optical methods (which use the luminous rays).

In order to increase the quality of the image, it is usual to administer some radiopaque contrast agents to the patient, which depends on the technique used and the route of administration. For example, one can note the use of oral barium sulfate suspension (for obtaining images of the digestive tract), injectable complexes, iron oxide particles, iodinated macromolecules or heavy metals like gadolinium or bismuth. In this last case, it is possible to enhance contrast considerably with these materials, which makes it possible to visualize the whole of arterial-venous network, various tissues or organs.

Although many chemical substances show contrasting effects, there will always exists a real interest for galenical chemical substances or dosage forms which will make it possible to target or visualize a specific organ/tissue and also an interest for substances with a very high contrasting effect in order to limit the quantities administered orally or in injectable way to the human or animal body. In addition, in margin of these two main interests, there exists a real need for contrasting substances having a strong vascular remanence, i.e. substances which could be eliminated slowly from the human organism or animal and for which a visualization of long life could be realized.

The patent application FR2868320 described a contrast agent for the MM characterized in that it comprises a complex including a chelating ligand and a metal ion of transition such as Mn, Co and Fe, the aforementioned ligand carrying a substituent whose electronic elimination or modification by chemical reaction or biochemical with a target substance causes a change of the state of the spin, and in particular of low spin to high spin.

The patent application FR2921837 described a new method of preparation of nanoparticles for the medical imaging including a metal core, an organic stabilizing layer and at least a ligand for the targeting of a pathological tissue.

The patent application FR2913886 refers to the formulation of metal nanoparticles covered with an organic protective coating for the diagnostic by MRI of Alzheimer's disease.

The patent EP0616538 refers to a new polyamine iodinated macromolecular compound, its preparation method and its use as a contrast agent. The importance of the iodine/macromolecule ratio makes it possible in this case to limit the concentration of contrast agent to be injected.

The patent application FR2921660 refers to hybrid inorganic/organic nanoparticles containing iron carboxylate which are usable as contrast agents and as a reservoir of active ingredients and therefore to allow the targeting of molecules such as pharmaceutically active drugs or markers for diagnosis.

However, important point to light out is generally the whole complexity of the chemistry involved in the formulations, which strongly limits the easy transposition of formulators in this specific field or even which brings some difficulties to potential industrial scale-ups and commercialization.

The inventors have developed an original formulation based on an extremely simple spontaneous emulsification of novel iodated amphiphiles leading to easy industrial scale-ups and commercialization.

The present invention refers to the compositions as well as a preparation method of injectable iodinated contrast agents for medical imaging. The iodinated contrast agents described in the present invention are amphiphilic molecules iodinated directly on the backbone of the lipophilic part of these amphiphilic molecules. We define “directly built-in” as being the result of the iodine fixing on a carbon atom by a covalent bond on a molecule comprising one or more double bonds, without fixing an additional aromatic groups like a benzene ring. Indeed, the iodine fixing on an amphiphilic molecule by a benzene ring was already asserted in various former patent applications (U.S. Pat. No. 4,957,729, U.S. Pat. No. 6,103,216, U.S. Pat. No. 7,582,279, WO 90/07491 and US 2009/311192).

The present invention concerns a iodinated amphiphilic compound of formula (I):

AG₁-X—R (I)

wherein

AG₁ represents an unsaturated (C₈-C₅₂) aliphatic hydrocarbon chain found in fatty acids having no double bond between the first and the second carbon atoms nor between the second and the third carbon atoms of each end of the chain, said chain comprising at least one iodine atom directly linked to a carbon atom of a non-conjugated double bond by a covalent link, the second carbon atom of the said double bond bearing a halogen atom different from a iodine atom,

X represents a polyethylene glycol comprising 1 to 50 ethylene glycol groups,

R represents a hydrogen atom or a group AG₂, AG₂ being an unsaturated (C₈-C₅₂) aliphatic hydrocarbon chain found in fatty acids having no double bond between the first and the second carbon atoms nor between the second and the third carbon atoms of each end of the chain, said chain comprising at least one iodine atom directly linked to a carbon atom of a non-conjugated double bond by a covalent link, the second carbon atom of the said double bond bearing a halogen atom different from an iodine atom, AG₂ being identical to or different from AG₁,

AG₁, X, R and AG₂ being selected so that the amphiphilic compound has a hydrophilic/lipohilic balance (HLB) comprised between 4 and 30.

In an advantageous embodiment, HLB is comprised between 4 and 16.

In an advantageous embodiment, AG₁ or AG₂ or both are an unsaturated (C₈-C₂₀) aliphatic hydrocarbon chain.

In another advantageous embodiment, X comprises 5 to 15 an unsaturated (C₈-C₅₂) aliphatic hydrocarbon chain.

According to the invention, the unsaturated (C₈-C₅₂) aliphatic hydrocarbon chains are issued from fatty acids which may be naturally occurring fatty acids or synthetic fatty acids. They are straight or branched carbon chains containing double and triple bonds between the carbon atoms.

In an advantageous embodiment of the invention, said are selected from the group comprising:
Alpha-linolenic acid (ALA) 18:3 (n-3) all-cis-9,12,15-octadecatrienoic acid,
Stearidonic acid (STD) 18:4 (n-3) all-cis-6,9,12,15,-octadecatetraenoic acid,
Eicosatrienoic acid (BE) 20:3 (n-3) all-cis-11,14,17-eicosatrienoic acid,
Eicosatetraenoic acid (ETA) 20:4 (n-3) all-cis-8,11,14,17-eicosatetraenoic acid,
Eicosapentaenoic acid (EPA) 20:5 (n-3) all-cis-5,8,11,14,17-eicosapentaenoic acid,
Docosapentaenoic acid (DPA, Clupanodonic acid) 22:5 (n-3) all-cis-7,10,13,16,19-docosapentaenoic acid,
Docosahexaenoic acid (DHA) 22:6 (n-3) all-cis-4,7,10,13,16,19-docosahexaenoic acid,
Tetracosapentaenoic acid 24:5 (n-3) Al-cis-9,12,15,18,21-tetracosapentaenoic acid,
Tetracosahexaenoic acid (Nisinic acid) 24:6 (n-3) Al-cis-6,9,12,15,18,21-tetracosahexaenoic acid,
Linoleic acid 18:2 (n-6) all-cis-9.12-octadecadienoic acid,
Gamma-linolenic acid (GLA) 18:3 (n-6) all-cis-6,9,12-octadecatrienoic acid,
Eicosadienoic acid 20:2 (n-6) all-cis-11.14-eicosadienoic acid,
Dihomo-gamma-linolenic acid (DGLA) 20:3 (n-6) all-cis-8,11,14-eicosatrienoic acid,
Arachidonic acid (AA) 20:4 (n-6) all-cis-5,8,11,14-eicosatetraenoic acid,
Docosadienoic acid 22:2 (n-6) all-cis-13.16-docosadienoic acid,
Adrenic acid 22:4 (n-6) all-cis-7,10,13,16-docosatetraenoic acid,
Docosapentaenoic acid (Osbond acid) 22:5 (n-6) all-cis-4,7,10,13,16-docosapentaenoic acid,
Oleic acid 18:1 (n-9) cis-9-octadecenoic acid,
Eicosenoic acid 20:1 (n-9) cis-11-eicosenoic acid,
Mead acid 20:3 (n-9) all-cis-5,8,11-eicosatrienoic acid,
Erucic acid 22:1 (n-9) cis-13-docosenoic acid, and
Nervonic acid 24:1 (n-9) cis-15-tetracosenoic acid,

The amphiphilic compounds according to the present invention are defined as being compounds which have a chemical structure and groups with parts of different polarities (ex surfactant non-ionic, triglycerides . . . ).

The fixing of iodine halogen molecules of type I—X (with X=Cl or Br) on an amphiphilic molecule is realized using a simple addition of halogenures to double bonds known as reaction of Wijs or Hanus. The fixing of halogens on the lipophilic part of an amphiphilic molecule modifies its physico-chemical properties considerably. Indeed, a modification of the amphiphilic molecules results in a steric obstruction in the middle of the lipidic part. This implies an important modification of its physicochemical properties (i.e solubility, impact on the interfacial tension, emulsifying properties), and thus of its properties implied in the formulation processes. This is the reason why the formulation of an injectable liquid containing this type of molecule proves to be difficult and why the former patents asserted the presence of iodine atoms on benzene rings fixed at the end of lipophilic chains.

The present invention refers to the use of iodinated amphiphilic molecules described above for the realization of particular colloidal systems called nano-emulsions of the type oil in water (still called lipophilic/hydrophilic) prepared by “low energy” techniques.

Another object of the invention are colloidal systems containing at least an iodinated compound such as those described above and are formulated with the addition or not of additional iodinated amphiphilic molecules, and can have the following structures:
oil-in-water emulsions;
auto-assembled systems;
nano-precipitates systems.

Thus another object of the invention is a nano-emulsion comprising at least an iodinated compound as defined above.

The oil-in-water nano-emulsions according to the invention are characterized by biphasic formulations containing droplets of a nonmiscible phase to water dispersed in an aqueous phase, stabilized or not by additional amphiphilic molecules, having droplet diameters lower than 500 nm, advantageously comprised between 20 and 200 nm, more advantageously between 100 and 200 nm.

The auto-assembled systems are colloidal systems which are formed by miscible molecules in the aqueous dispersing phase, which will be assembled spontaneously to form particles having a diameter lower than 500 nm, advantageously comprised between 20 and 200 nm.

The nano-precipitates systems are colloidal systems which are formed by not-miscible molecules in the aqueous dispersing phase and which, after a controlled precipitation, will form particles having a diameter lower than 500 nm, advantageously comprised between 20 and 200 nm.

All the formulations according to the invention, (oil-in-water emulsions, auto-assembled systems and nano-precipitates systems, can be described from a composition point of view by the following general formula:
2 to 70% (w/v) of iodinated amphiphilic compound;
0 to 20% (w/v) of a surfactant soluble in oil;
0.5 to 5% (w/v) of an osmolarity adjuster;
0.5 to 5% (w/v) of a pH adjuster;
0 to 1% (w/v) of an anti-oxidant agent;
water qs 100%.
   expressed in weight versus the volume of the formulation.

According to the invention, a surfactant is a molecule the structure of which presents both hydrophilic part(s) and lipophilic part(s).

The solutions or colloidal suspensions according to the present invention show the following general characteristics:
Osmolarity ranging between 200 and 400 mOsm/L;
Dynamic viscosity ranging between 0.3 and 100 mPa·s;
pH ranging between 5 and 10,
Mean diameter of the particles comprised between 20 and 200 nm, advantageously between 100 and 200 nm.

According to the invention, any surfactant known in the art, which is soluble in oil may be used; for example Cremophor® ELP. pH adjuster, osmolarity adjuster and antioxidant agents are also well known from the one skilled in the art.

The methods of preparation of these colloidal forms will be carried out exclusively by “low-energy” processes. “Low-energy” processes of formulation are processes for which the stage of emulsification does not imply a consumption of a total energy higher than 50 MegaJ/m³. This limit, in term of energy per unit of volume, corresponds to the manufacturing of nano-emulsions by using equipment such as a high pressure homogeneisator. The processes of formulation by low-energy used according to the invention, are based on the use of the physicochemical properties of the compounds allowing their recovery by a method of spontaneous emulsification or a method of phase inversion by using the temperature. All these techniques are well known from the one skilled in the art.

The emulsification step is defined as being the step during which the energy (whatever the form used) is brought to the system with an aim to divide the dispersed phase making it possible to generate the emulsions droplets, i.e. to increase the interfacial surface between the two non miscible phases.

The present invention can be realizable on a technical point of view only if the fixing of iodine or of the halogenated derivative on the molecule is not done on a conjugated carbon-carbon bond and must be absolutely realized starting from the 3rd (included) carbon from a final end and from the 3rd (included) carbon of the other end.

Thus the following structure of fatty-acid excluded:

Case 1: double bonds at the end of the aliphatic chain

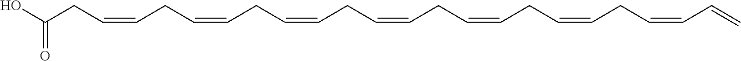 [SMILES: C=C/C=C\C/C=C\C/C=C\C/C=C\C/C=C\C/C=C\C/C=C\CC(=O)O]

Case 2: double bond combined with a carbonyl group

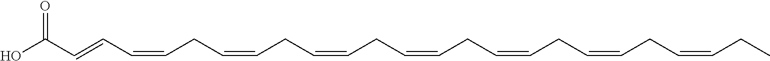 [SMILES: CC/C=C\C/C=C\C/C=C\C/C=C\C/C=C\C/C=C\C/C=C\C=C\C(=O)O]

The rate of fixing of iodine atoms by amphiphilic molecule is comprised between 1 and 14.
In a specific embodiment, the process for preparing an oil-in-water nanoemulsion according to the invention, comprises the following steps:
   contacting an amphiphilic compound according to the invention with a surfactant at a temperature comprised between 10 and 100° C., the ratio surfactant/oil (SOR) responding to the following equation:

SOR=100×w_{surfactant}/(w_{surfactant}+wₒᵢₗ),

with w_{surfactant} and wₒᵢₗ representing respectively the weight of surfactant and the weight if oil, adding the mixture obtained in step a) to an aqueous phase in order to have a ratio surfactant/oil/water (SOWR) responding to the following equation:

SOWR=100×w_{surfactant+oil}/(w_{surfactant+oil}+w_{water}).

SOWR being comprised between 30 and 50%, and w_{surfactant}, w_{water} and wₒᵢₗ representing respectively the weight of surfactant, the weight of water and the weight if oil,
optionally adjusting the osmolarity and/or the pH of the emulsion to be compatible with parenteral administration,
optionally sterilizing the nanoemulsion.
Another object of the invention is a composition comprising an effective radiological contrast producing amount of a radiological agent comprising a compound according to the invention and oil in water nanoemulsion as carrier for said radiological agent.
Still another object of the invention is a contrast agent for X-Ray imaging method comprising a nanoemulsion as disclosed above.
Another object of the invention is a X-Ray imaging method comprising the administration to a patient in need thereof of a nanoemulsion as disclosed above. In an advantageous embodiment, the method of X-Ray imaging comprises the steps of:

a) administering an imaging amount of the oil-in-water emulsion as defined above to a mammal and

b) when the imaging amount of the oil-in-water emulsion has reached the site to be imaged, carrying out X-Ray imaging of the site.

### DESCRIPTION OF THE FIGURES

FIG. 1 shows the influence of the value of the SOR on the diameter of the droplets and (insert) on the index of polydispersity, before (black squares) and after (white circles) iodation.

FIG. 2 illustrates the chemical structure of the iodinated amphiphilic compounds obtained according to example 1.

FIG. 3 illustrates the contrast enhancement obtained 75 min after IV injection. (A) Whole body coronal view crossing heart (h), liver (L) and bladder (b), showing the dual elimination routes. (B) Four cavities slice through the heart (obtained after reorientation), lines indicate positions of corresponding transverse slices (see below). (C) Transverse slice through the heart base: right atrium (ra), aortic valve plane (v), left atrium (la). (D) Transverse slice through the middle of the heart: right ventricle (rv) and left ventricle (lv). (E) Transverse slice through the apex of the heart. (F) Slice through the left ventricle showing the beginning of the aortic cross (a).

FIG. 4 illustrates the anatomical details of the kidneys vascularization at 75 min after IV injection: (A) Sagittal slice through the lombar spine, lines indicate position of corresponding coronal slices (see after). (B) Coronal slice through the dorsal part of the kidneys, showing the abdominal course of the aorta (aa) and the right and left dorsal muscular branches (respectively rdmd and ldmb). (C) Coronal slice 1.5 mm lower than (B) showing the right renal artery (ra). (D) Coronal slice 5 mm lower than (C) showing the venous side of kidney vascularization: left renal vein (rv) and left ovarian vein (ov), as well as the contrasted urine in the renal pelvis (rp).

FIG. 5 illustrates the scheme for the synthesis of iodinated amphiphilic compound containing docohexanoic acid according to example 2.

FIG. 6 illustrates the scheme for the synthesis of amphiphilic compound having 8 atoms of carbon according to example 3.

FIG. 7 illustrates the scheme for the synthesis of an amphiphilic compound containing arachidonic acid and having an hydrophilic group (PEG) of a molecular weight of 2000 Da according to example 4

FIG. 8 illustrates the scheme of the general synthesis of an amphiphilic monochain containing arachidonic acid and containing iodine and bromide according to example 5.

FIG. 9 illustrates the scheme of the general synthesis of amphiphilic monochain based on arachidonic acid and containing iodine and chloride according to example 5.

### EXAMPLES

### Example 1

### Protocol of Iodation 1:

The amphiphilic compound intended to be iodinated, is a compound called “Labrafil M1944CS” (1.5 g). This compound is solubilized in chloroform (100 ml). ICl (3 grams, 2 equivalents due to the number of double bonds) is added gently. The reaction medium is refluxed during 30 minutes. Then, the excess of ICl is neutralized by addition of a solution at 10% of KI (15 ml) and finally by the addition of sodium thiosulfate (% in water). The organic phase is then washed with water, dried on MgSO₄ and the solvent is evaporated under reduced pressure to obtain the amphiphilic compound on which is fixed covalently 2 atoms of Cl and 2 iodine atoms. The amphiphilic compound appears as a dark colored residue (output of 84%) Note: The addition of ICl on the double bond is not regioselective. The position of the various halogenous atoms of the diagrams is given only as an example.

### Protocol of Emulsification 1:

A step of mixture of the aforesaid amphiphilic compound iodinated (iodinated Labrafil M1944CS, noted as compound 1, of mass m1) with the not-iodinated amphiphilic compound (surfactant non-ionic Cremophor ELP, noted compound 2, of mass m2). The two compounds are totally miscible. This mixture is noted mixture A.

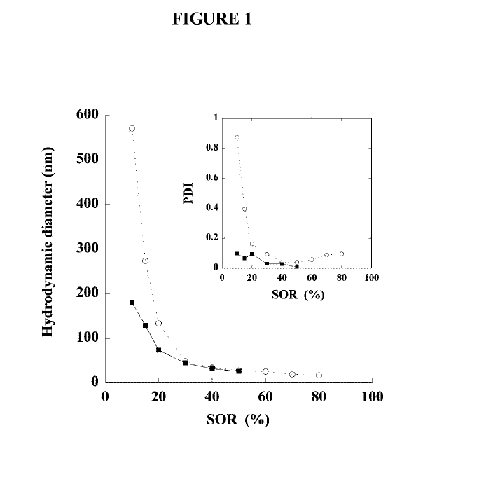

This mixture is characterized by a ratio of the weights SOR=100*m2/(m2+m1), and is fixed at 15%. The influence of the value of the SOR on the diameter of the droplets and the index of polydispersity of the nano-emulsions is given experimentally in the FIG. 1. Note: it can vary between 5% and 90% (included), which will affect the size of the obtained droplets.

The choice for this example is made on a value of SOR=15% which gives an average diameter of the droplets of 130 nm.

The mixture A is added to an aqueous phase constituted only of water. The proportions of mixture A/aqueous phase are fixed by the following relation (me=water mass): (m2+m1)/(m2+m1+me)=60. The colloidal system obtained in this case is a nano-emulsion which is formed spontaneously, by creating mixed droplets constituted of the compounds 1 and 2, dispersed in the aqueous phase.

Characterization of the Reaction Product. The Reaction Product is Characterized by Various Chemical Methods Well Known in the Art:

Elementary chemistry: Carbon: 50.70%; Hydrogen: 7.87%; Oxygen: 10.04%; Iodine: 24.53%; Chlorine: 6.86%.

NMR of the proton 1:00: (CDCl₃) δ 0.84 (T, 6:00, CH₃), δ 1.20 to 1.22 (m, 35H, other CH₂ of the fatty-acid), δ 1.48 (m, 4:00, CH₂ in β of groups COOR), δ 1.55 (m, 4:00, CH₂ in a of CHCl and CHI), δ 2.25 (T, 4:00, CH₂ of the fatty-acid in a of group COOR), δ 3.61 (m, 9:00, other CH₂ of the part of poly(ethylene glycol)), δ 4.08 (m, 6:00, CH₂ of the part poly(ethylene glycol) in a of groups COOR and CHCl), δ 4.45 (m, 1:00, CHI).

NMR of carbon 13C: (CDCl₃) δ 14.121 (C-18), δ 22.659 (C-17), δ 24.923 (C-3), δ 26.7 (C-7 and C-12), δ 31.0 (other C of the fatty-acid), δ 34.054 (C-2), δ 34.415 (C-8), δ 37.532 (C-11), δ 42.435 (C-9), δ 63.377 (C-1′), δ 65.604 (C-10), δ 70.4 (other C of the groups poly(ethylene glycol)), δ 173.721 (C-1).

Spectroscopy of high-resolution mass (ES+): there are two products prevailing which present a Gauss distribution, which corresponds to three times 44 Da (which correspond to a monomeric ethylene glycol) on the two sides m/z 1217.39497 (M+K+, 15.58) and 791.27352 (M+K+, 84.42).

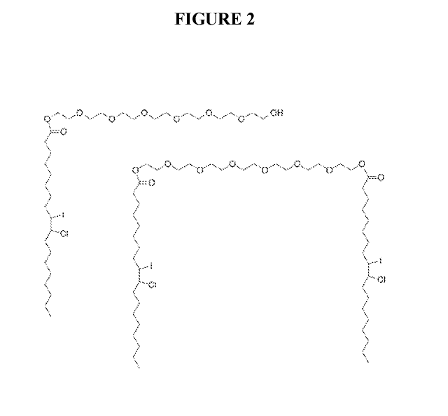

These results lead to the structure presented on FIG. 2 for the amphiphilic compound iodinated in this example.

### Example of Application: In Vivo Evaluation of the Formulated Nano-Emulsions

The colloidal systems (100 μl containing 8% iodine w/w) were injected intravenously in nude mice.

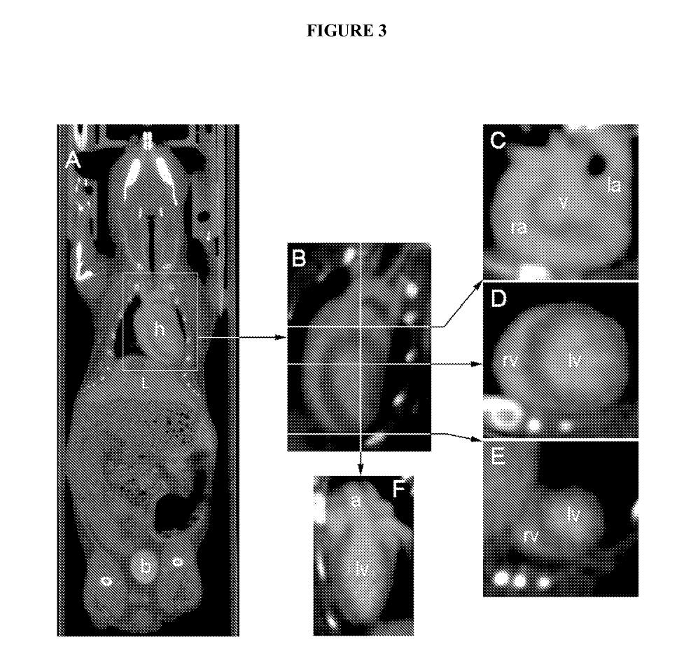

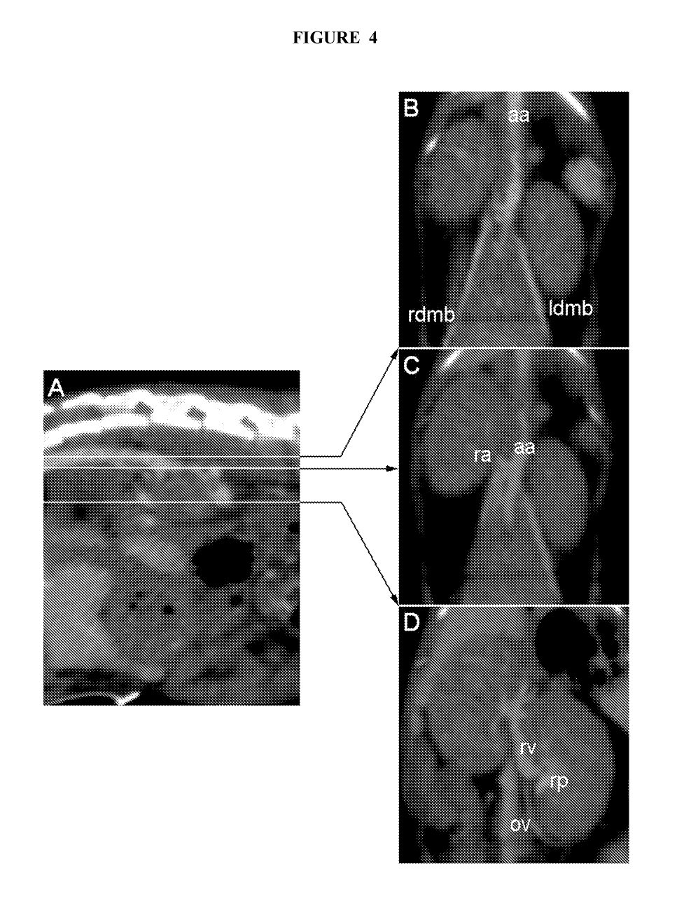

The results presented on FIGS. 3 and 4 for a SOR of 15% and a SOWR=40%, sizing at 120 nm.

The iodinated nano-emulsions selected described above (SOR=15% and SOWR=40%, sizing at 120 nm) were intravenously injected in mice. The results presented in FIGS. 3 and 4 show pictures acquired 75 min after the nano-emulsion injection, and are focused on heart and kidney, respectively. A clear vascular contrast is obtained which allows obtaining clear images of the blood pool. They definitively show the potentials of such iodinated nano-emulsions as blood pool contrast agents for preclinical imaging by disclosing their significant X-ray attenuation power.

### Example 2

### Synthesis of Iodinated Amphiphilic Compound Resulting from the Docosahedric Acid

### Protocol of Synthesis and Iodation 2:

Thionyl chloride (1.4 grams) in solution in anhydrous dichloromethane (10 ml) is added drop by drop to a docosahedric acid solution (3.28 grams) in anhydrous dichloromethane (100 ml). After 30 minutes of agitation, a solution of polyethylene glycol of average molecular mass of 400 (2 grams) is added in dichloromethane (20 ml) and the reaction medium is agitated during the night to give place to the formation of ester bonds. The organic phase is then washed with water, dried on MgSO₄ and the solvent is evaporated under reduced pressure. The residue is then solubilized by using chloroform (100 ml). ICl (10 grams, 6 equivalents calculated in function of the number of double bonds) is added gently. The reaction medium is refluxed during 30 minutes. The excess of ICl is neutralized by addition of a solution at 10% of KI (15 ml) and finally by sodium thiosulfate (% in water).

The organic phase is then washed with water, dried on MgSO₄ and the solvent is evaporated under reduced pressure to obtain the amphiphilic compound on which are attached 12 chlorine atoms and 12 iodine atoms. The amphiphilic compound appears as a dark residue colored (11.8 grams, rate of 80%) Note: The addition of ICl on the double bond is not regioselective. The position of the different halogenous atoms of the diagrams is given only as illustration.

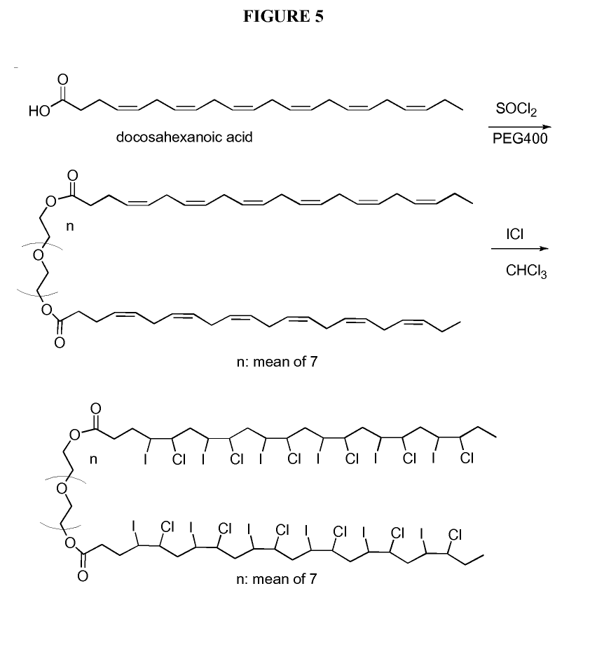

The scheme for the synthesis is given in FIG. 5.

### Protocol of Emulsification 2:

A step of mixture of the aforesaid amphiphilic iodinated compound containing docohexanoic acid is carried out with the non-iodinated amphiphilic compound (non ionic surfactant like Cremophor ELP). The two compounds are perfectly miscible.

This mixture is characterized by a ratio of the weights noted SOR fixed at 15%. The diameter of the droplets obtained is of 102 nm. This mixture is added to an aqueous phase made up of only water. As for example 1, the proportions of the mixture/aqueous phase are fixed at 60. In this case, the obtained colloidal system is a nano-emulsion which is obtained spontaneously, by creating mixed droplets made up by the iodinated and not-iodinated amphiphilic compounds, dispersed in the aqueous phase.

### Example 3

### Synthesis of Iodinated Amphiphile Resulting from the Octaenoic Acid

### Protocol of Synthesis 3:

ICl (18 grams), is added gently to an octaenoic acid solution (14 grams) in dichloromethane (400 ml). The reaction medium is refluxed during 30 minutes. The excess of ICl is neutralized by addition of a solution at 10% of KI (15 ml) and finally by sodium thiosulfate (% in water). The organic phase is then washed with water, dried on MgSO₄ and the solvent is evaporated under reduced pressure. The residue (the acid iodochloro octanoic) is then solubilized in anhydrous methylene chloride (400 ml) and thionyl chloride (14 grams) in solution in anhydrous methylene chloride (30 ml) and added drop by drop. After 30 minutes of agitation, a solution of polyethylene glycol of an average molecular weight of 400 (20 grams) in methylene chloride (40 ml) is added and the reaction medium is agitated during the night in order to obtain the formation of ester bonds. The organic phase is then washed with water, dried on MgSO₄ and the solvent is evaporated under reduced pressure to obtain the amphiphilic compound comprising 8 atoms of carbon, each one having fixed covalently 1 atom of chloride and 1 atom of iodine. The obtained amphiphilic compound appears as a colored residue (rate of 70%).

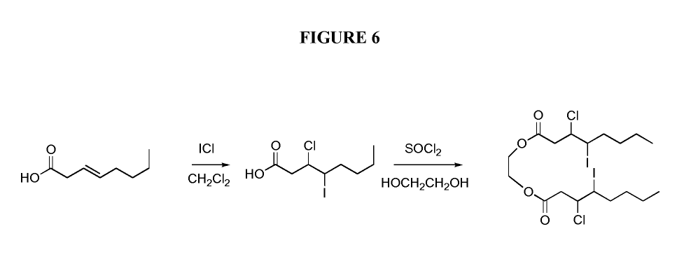

The scheme of the synthesis is given in FIG. 6.

### Protocol of Emulsification 3:

A step of mixture of the aforesaid iodinated amphiphilic compound synthesized above is realized with the non iodinated amphiphilic compound (non ionic surfactant like Cremophor ELP type). The two compounds are completely miscible.

This mixture is characterized by a ratio of the weights noted SOR and fixed at 15%, the diameter of the droplets obtained is of 173 nm. This mixture is added to an aqueous phase containing only water. As for example 1, the proportions of the mixture/aqueous phase are fixed at 60. The obtained colloidal system in this case is a nano-emulsion which is formed spontaneously, by creating mixed droplets constituted of iodinated and non iodinated amphiphilic compounds, dispersed in the aqueous phase.

### Example 4

### Synthesis of an Amphiphilic Compound Containing Arachidonic Acid and Having an Hydrophilic Group (PEG) of a Molecular Weight of 2000 Da

### Protocol of Synthesis 4:

Thionyl chloride (1.4 grams) in solution in anhydrous methylene chloride (10 ml) is added drop by drop to an arachidonic acid solution (3.0 grams) in anhydrous methylene chloride (100 ml). After 30 minutes of agitation, a solution of polyethylene glycol of an average molecular weight of 2000 (10 grams) in methylene chloride (20 ml) is added and the reaction medium is agitated during the night in order to obtain the formation of ester bonds.

The organic phase is then washed with water, dried on MgSO₄ and the solvent is evaporated under reduced pressure. The residue is then solubilized in chloroform (100 ml). ICl (6.4 grams) is added gently. The reaction medium is refluxed during 30 minutes. Then, the excess of ICL is neutralized by addition of a solution at 10% of KI (15 ml) and finally by sodium thiosulfate (% in water).

The organic phase is then washed with water, dried on MgSO₄ and the solvent is evaporated under reduced pressure to obtain the amphiphilic compound on which were fixed covalently 8 atoms of chlorine and 8 atoms of iodine. The amphiphilic compound appears as a dark colored residue (rate of 65%).

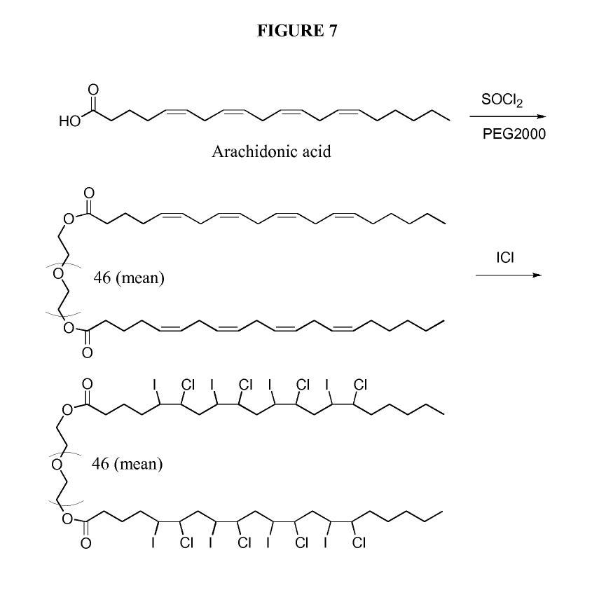

The scheme of the synthesis is illustrated in FIG. 7.

### Protocol of Emulsification 4:

A step of mixture of the aforesaid iodinated amphiphilic compound based on arachidonic acid is realized with the non iodinated amphiphilic compound (non ionic surfactant like Cremophor ELP). The two compounds are completely miscible.

This mixture is characterized by a ratio of the weights noted as SOR and fixed at 15%, the diameter of the drops obtained is of 119 nm. This mixture is added to an aqueous phase (deionized water). As for example 1, the proportions of the mixture/aqueous phase are fixed at 60. In this case, the resulting colloidal system is a nano-emulsion which is formed spontaneously, by creating mixed droplets made up by the iodinated and non iodinated amphiphilic compounds, dispersed in the aqueous phase.

### Example 5

### Synthesis of an Amphiphilic Monochain Compound Containing Arachidonic Acid

### Protocol of Synthesis 5:

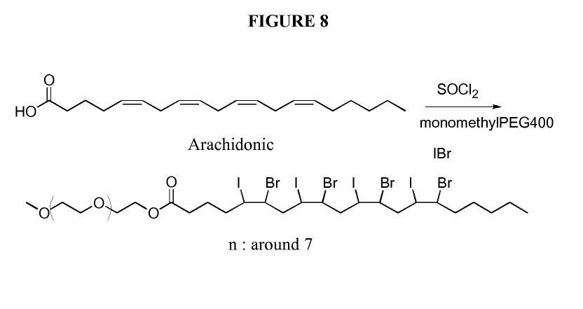

The general protocol is given in FIG. 8.

Thionyl chloride (1.4 grams) in solution in anhydrous methylene chloride (10 ml) is added drop by drop to an arachidonic acid solution (3.0 grams) in anhydrous methylene chloride (100 ml). After 30 minutes of agitation, a solution of monomethylpolyethylene glycol of an average molecular weight of 400 (5 grams) in methylene chloride (20 ml) is added and the reaction medium is agitated during the night to give the formation of ester bond. The organic phase is then washed with water, dried on MgSO₄ and the solvent is evaporated under reduced pressure. The residue is then solubilized in chloroform (100 ml). IBr (8.2 grams) is added gently. The reaction medium is refluxed during 4 hours. The excess of IBr is neutralized by addition of a solution at 10% of KI (15 ml) and finally with sodium thiosulfate (% in water).

The organic phase is then washed with water, dried on MgSO₄ and the solvent is evaporated under reduced pressure to obtain the amphiphilic compound having fixed covalently 4 bromide atoms and 4 iodine atoms. The amphiphilic compound appears as a dark colored residue (rate of 65%).

### Protocol of Emulsification 5:

A step of mixture of the aforesaid iodinated/brominated amphiphilic monochain compound containing arachidonic acid is realized with the non iodinated amphiphilic compound (Labrafil® M1944CS). The two compounds are completely miscible.

This mixture is characterized by a ratio of the weights noted as SOR and fixed at 50%, the diameter of the droplets obtained is of 60 nm. This mixture is added to an aqueous phase (deionized water). As for example 1, the proportions of the mixture/aqueous phase are fixed at 60. In this case, the obtained colloidal system is a nano-emulsion which is formed spontaneously, by creating mixed droplets constituted of the iodinated and non iodinated amphiphilic compounds, dispersed in the aqueous phase.

### Example 6

### Synthesis of an Amphiphilic Monochain Compound Based on Arachidonic Acid

### Protocol of Synthesis 6:

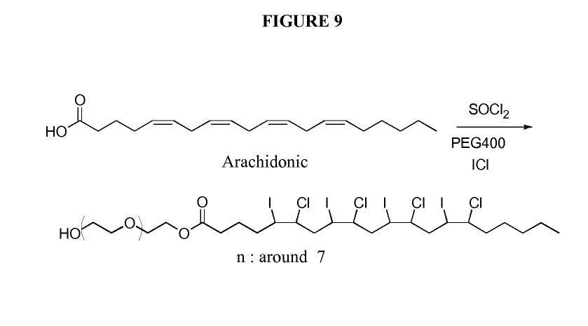

The general scheme is given in FIG. 9.

Thionyl chloride (1.4 grams) in solution in anhydrous methylene chloride (10 ml) is added drop by drop to a solution of arachidonic acid (3.0 grams) in anhydrous methylene chloride (100 ml). After 30 minutes of agitation, a solution of polyethylene glycol of an average molecular weight of 400 (10 grams) in methylene chloride (20 ml) is added and the reaction medium is agitated during the night in order to form ester bonds. The organic phase is then washed with water, dried on MgSO₄ and the solvent is evaporated under reduced pressure. The residue is then solubilized in chloroform (100 ml). ICl (7.0 grams) is added gently. The reaction medium is refluxed during 4 hours. The excess of ICl is neutralized by addition of a solution at 10% of KI (15 ml) and finally by addition of sodium thiosulfate (% in water).

The organic phase is then washed with water, dried on MgSO₄ and the solvent is evaporated under reduced pressure in order to obtain the amphiphilic compound on which are fixed covalently 4 chlorine atoms and 4 iodine atoms. The amphiphilic compound appears as a colored residue (rate of 70%).

### Protocol of Emulsification 6:

A step of mixture of the aforesaid iodinated amphiphilic monochain compound containing arachidonic acid is realized with the non iodinated amphiphilic compound (Labrafil® M1944CS). The two compounds are completely miscible.

This mixture is characterized by a ratio of the weights noted as SOR fixed at 50%, the diameter of the droplets obtained is of 84 nm. This mixture is added to an aqueous phase (deionized water). As for example 1, the proportions of the mixture/aqueous phase are fixed at 60. In this case, the colloidal system is a nano-emulsion which is formed spontaneously, by creating mixed droplets constituted by the iodinated and not-iodinated amphiphilic compounds, dispersed in the aqueous phase.

1. Iodinated amphiphilic compound of formula (I):
AG₁-X—R (I)
wherein
AG₁ represents an unsaturated (C₈-C₅₂) aliphatic hydrocarbon chain found in fatty acids having no double bond between the first and the second carbon atoms nor between the second and the third carbon atoms of each end of the chain, said chain comprising at least one iodine atom directly linked to a carbon atom of a non-conjugated double bond by a covalent link, the second carbon atom of the said double bond bearing a halogen atom different from a iodine atom,
X represents a polyethylene glycol comprising 1 to 50 ethylene glycol groups,
R represents a hydrogen atom or a group AG₂, AG₂ being an unsaturated (C₈-C₅₂) aliphatic hydrocarbon chain found in fatty acids having no double bond between the first and the second carbon atoms nor between the second and the third carbon atoms of each end of the chain, said chain comprising at least one iodine atom directly linked to a carbon atom of a non-conjugated double bond by a covalent link, the second carbon atom of the said double bond bearing a halogen atom different from an iodine atom, AG₂ being identical to or different from AG₁,
AG₁, X, R and AG₂ being selected so that the amphiphilic compound has a hydrophilic/lipohilic balance comprised between 4 and 30.

2. Compounds according to claim 1 wherein AG₁ or AG₂ or both are an unsaturated (C₈-C₂₀) aliphatic hydrocarbon chain.

3. Compounds according to claim 1 wherein the X comprises 5 to 15 an unsaturated (C₈-C₅₂) aliphatic hydrocarbon chain.

4. Compounds according to claim 1 wherein AG₁ or AG₂ are independently selected from the group comprising: alpha-linolenic acid (ALA) 18:3 (n-3) all-cis-9,12,15-octadecatrienoic acid, Stearidonic acid (STD) 18:4 (n-3) all-cis-6,9,12,15,-octadecatetraenoic acid, eicosatrienoic acid (ETE) 20:3 (n-3) all-cis-11,14,17-eicosatrienoic acid, eicosatetraenoic acid (ETA) 20:4 (n-3) all-cis-8,11,14,17-eicosatetraenoic acid, eicosapentaenoic acid (EPA) 20:5 (n-3) all-cis-5,8,11,14,17-eicosapentaenoic acid, docosapentaenoic acid (DPA, Clupanodonic acid) 22:5 (n-3) all-cis-7,10,13,16,19-docosapentaenoic acid, docosahexaenoic acid (DHA) 22:6 (n-3) all-cis-4,7,10,13,16,19-docosahexaenoic acid, tetracosapentaenoic acid 24:5 (n-3) all-cis-9,12,15,18,21-tetracosapentaenoic acid, tetracosahexaenoic acid (Nisinic acid) 24:6 (n-3) all-cis-6,9,12,15,18,21-tetracosahexaenoic acid, linoleic acid 18:2 (n-6) all-cis-9,12-octadecadienoic acid, gamma-linolenic acid (GLA) 18:3 (n-6) all-cis-6,9,12-octadecatrienoic acid, eicosadienoic acid 20:2 (n-6) all-cis-11,14-eicosadienoic acid, dihomo-gamma-linolenic acid (DGLA) 20:3 (n-6) all-cis-8,11,14-eicosatrienoic acid, arachidonic acid (AA) 20:4 (n-6) all-cis-5,8,11,14-eicosatetraenoic acid, docosadienoic acid 22:2 (n-6) all-cis-13,16-docosadienoic acid, adrenic acid 22:4 (n-6) all-cis-7,10,13,16-docosatetraenoic acid, docosapentaenoic acid (Osbond acid) 22:5 (n-6) all-cis-4,7,10,13,16-docosapentaenoic acid, oleic acid 18:1 (n-9) cis-9-octadecenoic acid, eicosenoic acid 20:1 (n-9) cis-11-eicosenoic acid, mead acid 20:3 (n-9) all-cis-5,8,11-eicosatrienoic acid, erucic acid 22:1 (n-9) cis-13-docosenoic acid and nervonic acid 24:1 (n-9) cis-15-tetracosenoic acid.

5. Oil in water nanoemulsion comprising a iodinated compound according to claim 1.

6. Oil in water emulsion according to claim 5 comprising:
2 to 70% (w/v) of at least one iodinated compound,
0 to 20% (w/v) of a surfactant soluble in oil,
0.5 to 5% (w:v) of an osmolarity adjuster,
0.5 to 5% (w/v) of a pH adjuster,
0 to 1% (w/v) antioxidant and
Water qs 100%
expressed in weight versus the volume of nanoemulsion.

7. Oil in water nanoemulsion according to claim 6 having the following features:
an osmolarity comprised between 200 and 400 mOsm/l.
a dynamic viscosity comprised between 0.3 et 100 mPa·s
a pH comprised between 5 and 10 and
a mean diameter of the particles comprised between 20 and 200 nm.

8. Process for preparing an oil in water nanoemulsion according to claim 5 comprising the following steps:
a) contacting an amphiphilic compound with a surfactant soluble in oil at a temperature comprised between 10 and 100° C., the ratio surfactant/oil (SOR) responding to the following equation:
SOR=100×w_{surfactant}/(w_{surfactant}+wₒᵢₗ),
b) adding the mixture obtained in step a) to an aqueous phase in order to have a ratio surfactant/oil/water (SOWR) responding to the following equation:
SOWR=100×w_{surfactant+oil}/(w_{surfactant+oil}+w_{water}).
SOWR being comprised between 30 and 50%,
c) optionally adjusting the osmolarity and/or the pH of the emulsion to be compatible with parenteral administration and
d) optionally sterilizing the nanoemulsion.

9. Composition comprising an effective radiological contrast producing amount of a radiological agent comprising a compound according to claim 1 and oil in water nanoemulsion as carrier for said radiological agent.

10. Contrast agent for X-Ray imaging method comprising a nanoemulsion according to claim 5.

11. X-Ray imaging method comprising the administration to a patient in need thereof a nanoemulsion according to claim 5.

12. A method of X-Ray imaging comprising the steps of:
a) administering an imaging amount of the oil-in-water emulsion of claim 5 to a mammal wherein the oil-in-water emulsion and
b) when the imaging amount of the oil-in-water emulsion has reached the site to be imaged, carrying out X-Ray imaging of the site.
